(19) United States
(12) Patent Application Publication
(10) Pub. No.: US 2002/0107379 A1
(43) Pub. Date: Aug. 8, 2002

(54) Nucleotide sequences coding for the thyA gene

(76) Inventors: Achim Marx, Bielefeld (DE); Natalie Schischka, Bielefeld (DE); Brigitte Bathe, Salzkotten (DE); Mike Farwick, Bielefeld (DE)

Correspondence Address: SMITH, GAMBRELL & RUSSELL, LLP, 1850 M STREET, N.W., SUITE 800, WASHINGTON, DC, 20036, US

(21) Appl. No.: 09/954,197

(22) Filed: Sep. 18, 2001

(30) Foreign Application Priority Data
Sep. 20, 2000 (DE) 100 46 626.5
Jul. 7, 2001 (DE) 101 33 162.2

(51) Int. Cl. C12P 13/08; C07H 21/04; C12N 9/10; C12N 1/21; C12P 21/02

(52) U.S. Cl. 536/23.2; 435/115; 435/69.1; 435/252.3; 435/320.1; 435/193

(57) ABSTRACT

The invention relates to an isolated polynucleotide having a polynucleotide sequence which codes for the thyA gene, and a host-vector system having a coryneform host bacterium in which the thyA gene is present in attenuated form and a vector which carries at least the thyA gene according to SEQ ID No 1, and the use of polynucleotides which comprise the sequences according to the invention as hybridization probes

### BACKGROUND OF THE INVENTION

The invention provides nucleotide sequences from coryneform bacteria coding for the thyA gene, and a process for the production of amino acids by fermentation using bacteria in which the thyA gene is enhanced. All references cited herein are expressly incorporated by reference. Incorporation by reference is also designated by the term “I.B.R.” following any citation.

L-amino acids, especially L-lysine, are used in human medicine and in the pharmaceuticals industry, in the foodstuffs industry and, very especially, in the feeding of animals.

It is known that amino acids are produced by fermentation of strains of coryneform bacteria, especially Corynebacterium glutamicum. Because of their great importance, attempts are continuously being made to improve the production processes. Improvements to the processes may concern measures relating to the fermentation, such as, for example, stirring and oxygen supply, or the composition of the nutrient media, such as, for example, the sugar concentration during the fermentation, or working up to the product form by, for example, ion-exchange chromatography, or the intrinsic performance properties of the microorganism itself.

In order to improve the performance properties of such microorganisms, methods of mutagenesis, selection and mutant selection are employed. Such methods yield strains which are resistant to antimetabolites or are auxotrophic for metabolites that are important in terms of regulation, and which produce amino acids.

For a number of years, methods of recombinant DNA technology have also been used for improving the strain of L-amino acid-producing strains of Corynebacterium, by amplifying individual amino acid biosynthesis genes and studying the effect on amino acid production.

The invention provides novel measures for the improved production of amino acids by fermentation.

### BRIEF SUMMARY OF THE INVENTION

Where L-amino acids or amino acids are mentioned hereinbelow, they are to be understood as meaning one or more amino acids, including their salts, selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-lysine is particularly preferred.

Where L-lysine or lysine is mentioned hereinbelow, it is to be understood as meaning not only the bases but also the salts, such as, for example, lysine monohydrochloride or lysine sulfate.

The invention provides an isolated polynucleotide from coryneform bacteria, containing a polynucleotide sequence coding for the thyA gene, selected from the group

a) polynucleotide that is at least 70% identical with a polynucleotide that codes for a polypeptide containing the amino acid sequence of SEQ ID No. 2,

b) polynucleotide that codes for a polypeptide containing an amino acid sequence that is at least 70% identical with the amino acid sequence of SEQ ID No. 2,

c) polynucleotide that is complementary to the polynucleotides of a) or b), and

d) polynucleotide containing at least 15 consecutive nucleotides of the polynucleotide sequence of a), b) or c),

the polypeptide preferably exhibiting the activity of thymidilate synthase.

The invention also provides the above-mentioned polynucleotide, it preferably being a replicable DNA containing:

(i) the nucleotide sequence shown in SEQ ID No. 1, or

(ii) at least one sequence that corresponds to sequence (i) within the region of the degeneracy of the genetic code, or

(iii) at least one sequence that hybridizes with the sequence that is complementary to sequence (i) or (ii), and optionally

(iv) sense mutations in (i) that are neutral in terms of function.

The invention also provides

a replicable polynucleotide, especially DNA, containing the nucleotide sequence as shown in SEQ ID No. 1;

a polynucleotide that codes for a polypeptide containing the amino acid sequence as shown in SEQ ID No. 2;

a vector containing the polynucleotide of the invention, especially a shuttle vector or a plasmid vector, and

coryneform bacteria which contain the vector or in which the thyA gene has been enhanced.

The invention also provides polynucleotides consisting essentially of a polynucleotide sequence, which are obtainable by screening, by means of hybridization, a corresponding gene library of a coryneform bacteria that contains the complete gene or parts thereof, using a probe containing the sequence of the polynucleotide of the invention according to SEQ ID No. 1 or a fragment thereof, and isolating the mentioned polynucleotide sequence.

### BRIEF DESCRIPTION OF THE FIGURES

FIG. 1: Map of plasmid pEC-XK99E

FIG. 2: Map of plasmid pEC-XK99EthyAb1ex

The abbreviations and names used have the following meanings:

| | |
|---|---|
| Kan: | Kanamycin resistance gene aph(3′)-IIa from |
| | Escherichia coli |
| HindIII | cleavage site of the restriction enzyme |
| | HindIII |
| XbaI | cleavage site of the restriction enzyme XbaI |
| KpnI | cleavage site of the restriction enzyme KpnI |
| Ptrc | trc promoter |
| T1 | termination region T1 |
| T2 | termination region T2 |
| Per | replication effector per |
| Rep | replication region rep of plasmid pGA1 |
| LacIq | lacIq repressor of the lac operon of |
| | Escherichia coli |
| ThyA | cloned thyA gene |

### DETAILED DESCRIPTION OF THE INVENTION

Polynucleotides that contain the sequences of the invention are suitable as hybridization probes for RNA, cDNA and DNA, in order to isolate in their complete length nucleic acids, or polynucleotides or genes, that code for thymidilate synthase, or in order to isolate nucleic acids, or polynucleotides or genes, that are very similar to the sequence having the thyA gene. They are likewise suitable for incorporation into so-called arrays, micro arrays or DNA chips in order to detect and determine the corresponding polynucleotides.

Polynucleotides that contain the sequences of the invention are also suitable as primers, with the aid of which it is possible, by means of the polymerase chain reaction (PCR), to produce DNA of genes that code for thymidilate synthase.

Such oligonucleotides acting as probes or primers contain at least 25, 26, 27, 28, 29 or 30, preferably at least 20, 21, 22, 23 or 24, most particularly preferably at least 15, 16, 17, 18 or 19, consecutive nucleotides. Also suitable are oligonucleotides having a length of at least 31, 32, 33, 34, 35, 36, 37, 38, 39 or 40 or of at least 41, 42, 43, 44, 45, 46, 47, 48, 49 or 50 nucleotides. Oligonucleotides having a length of at least 100, 150, 200, 250 or 300 nucleotides may also be suitable.

“Isolated” means removed from its natural environment.

“Polynucleotide” generally refers to polyribonucleotides and polydeoxyribonucleotides, it being possible for the RNA or DNA to be unmodified or modified.

The polynucleotides of the invention include a polynucleotide according to SEQ ID No. 1 or a fragment prepared therefrom, and also polynucleotides that are at least from 70% to 80%, preferably at least from 81% to 85%, particularly preferably at least from 86% to 90%, and most particularly preferably at least 91%, 93%, 95%, 97% or 99%, identical with the polynucleotide according to SEQ ID No. 1, or a fragment prepared therefrom.

“Polypeptides” are to be understood as being peptides or proteins that contain two or more amino acids bonded via peptide bonds.

The polypeptides of the invention include a polypeptide according to SEQ ID No. 2, especially those having the biological activity of thymidilate synthase, and also those that are at least from 70% to 80%, preferably at least from 81% to 85%, particularly preferably at least from 86% to 90%, and most particularly preferably at least 91%, 93%, 95%, 97% or 99%, identical with the polypeptide according to SEQ ID No. 2 and exhibit the mentioned activity.

The invention also provides a process for the production, by fermentation, of amino acids selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine, using coryneform bacteria which, in particular, already produce amino acids and in which the nucleotide sequences coding for the thyA gene are enhanced, especially overexpressed.

The term “enhancement” in this context describes an increase in the intracellular activity of one or more enzymes (proteins) in a microorganism that are coded for by the corresponding DNA, by, for example, increasing the number of copies of the gene or genes, using a strong promoter or using a gene or allele that codes for a corresponding enzyme (protein) having a high degree of activity, and optionally by combining those measures.

By the measures of enhancement, especially overexpression, the activity or concentration of the corresponding protein is generally increased by at least 10%, 25%, 50%, 75%, 100%, 150%, 200%, 300%, 400% or 500%, at most by up to 1000% or 2000%, based on that of the starting microorganism.

The microorganisms provided by the present invention can produce L-amino acids from glucose, saccharose, lactose, fructose, maltose, molasses, starch, cellulose or from glycerol and ethanol. They may be representatives of coryneform bacteria, especially of the genus Corynebacterium. In the case of the genus Corynebacterium, special mention may be made of the species Corynebacterium glutamicum, which is known to those skilled in the art for its ability to produce L-amino acids.

Suitable strains of the genus Corynebacterium, especially of the species Corynebacterium glutamicum (C. glutamicum), are especially the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC14067

Brevibacterium lactofermentum ATCC13869 and

Brevibacterium divaricatum ATCC14020

and L-amino acid-producing mutants or strains prepared therefrom.

The new thyA gene of C. glutamicum coding for the enzyme thymidilate synthase (EC 2.1.1.45) has been isolated.

In order to isolate the thyA gene or other genes from C. glutamicum, a gene library of that microorganism in Escherichia coli (E. coli) is first prepared. The preparation of gene libraries is described in generally known textbooks and handbooks. There may be mentioned as an example the textbook of Winnacker: Gene und Klone, Eine Einführung in die Gentechnologie (Verlag Chemie, Weinheim, Germany, 1990 I.B.R.) or the handbook of Sambrook et al.: Molecular Cloning, A Laboratory Manual (Cold Spring Harbor Laboratory Press, 1989) I.B.R. A very well known gene library is that of the E. coli K-12 strain W3110, which has been prepared by Kohara et al. (Cell 50, 495-508 (1987)) I.B.R. in λ-vectors. Bathe et al. (Molecular and General Genetics, 252:255-265, 1996) I.B.R. describe a gene library of C. glutamicum ATCC13032, which has been prepared with the aid of the cosmid vector SuperCos I (Wahl et al., 1987, Proceedings of the National Academy of Sciences USA, 84:2160-2164 I.B.R.) in the E. coli K-12 strain NM554 (Raleigh et al., 1988, Nucleic Acids Research 16:1563-1575 I.B.R.).

Börmann et al. (Molecular Microbiology 6(3), 317-326 (1992) I.B.R.) in turn describe a gene library of C. glutamicum ATCC13032 using the cosmid pHC79 (Hohn and Collins, Gene 11, 291-298 (1980) I.B.R.).

For the preparation of a gene library of C. glutamicum in E. coli it is also possible to use plasmids such as pBR322 (Bolivar, Life Sciences, 25, 807-818 (1979) I.B.R.) or pUC9 (Vieira et al., 1982, Gene, 19:259-268 I.B.R.). Suitable hosts are especially those E. coli strains that are restriction- and recombination-defective. An example thereof is the strain DH5αmcr, which has been described by Grant et al. (Proceedings of the National Academy of Sciences USA, 87 (1990) 4645-4649) I.B.R. The long DNA fragments cloned with the aid of cosmids can then in turn be subcloned into customary vectors suitable for the sequencing and then sequenced, as is described, for example, in Sanger et al. (Proceedings of the National Academy of Sciences of the United States of America, 74:5463-5467, 1977) I.B.R.

The resulting DNA sequences can then be studied using known algorithms or sequence-analysis programs, such as, for example, that of Staden (Nucleic Acids Research 14, 217-232 (1986)) I.B.R., that of Marck (Nucleic Acids Research 16, 1829-1836 (1988)) I.B.R. or the GCG program of Butler (Methods of Biochemical Analysis 39, 74-97 (1998)) I.B.R.

The novel DNA sequence of C. glutamicum coding for the thyA gene has been found and, as SEQ ID No. 1, forms part of the present invention. Furthermore, the amino acid sequence of the corresponding protein has been derived from the present DNA sequence using the methods described above. The resulting amino acid sequence of the thyA gene product is shown in SEQ ID No. 2.

Coding DNA sequences that result from SEQ ID No. 1 by the degeneracy of the genetic code also form part of the invention. Likewise, DNA sequences that hybridize with SEQ ID No. 1 or parts of SEQ ID No. 1 form part of the invention. Furthermore, to those skilled in the art, conservative amino acid substitutions, such as, for example, the substitution of glycine with alanine or of aspartic acid with glutamic acid, in proteins are known as sense mutations, which do not lead to any fundamental change in the activity of the protein, that is to say are neutral in terms of function. It is also known that changes at the N- and/or C-terminus of a protein do not substantially impair its function or may even stabilize it. The person skilled in the art will find relevant information inter alia in Ben-Bassat et al. (Journal of Bacteriology 169:751-757 (1987)) I.B.R., in O'Regan et al. (Gene 77:237-251 (1989)) I.B.R., in Sahin-Toth et al. (Protein Sciences 3:240-247 (1994)) I.B.R., in Hochuli et al. (Bio/Technology 6:1321-1325 (1988)) I.B.R. and in known textbooks of genetics and molecular biology. Amino acid sequences that result in a corresponding manner from SEQ ID No. 2 likewise form part of the invention.

Similarly, DNA sequences that hybridize with SEQ ID No. 1 or parts of SEQ ID No. 1 form part of the invention. Finally, DNA sequences that are produced by the polymerase chain reaction (PCR) using primers that result from SEQ ID No. 1 form part of the invention. Such oligonucleotides typically have a length of at least 15 nucleotides.

The person skilled in the art will find instructions on the identification of DNA sequences by means of hybridization inter alia in the handbook “The DIG System Users Guide for Filter Hybridization” from Boehringer Mannheim GmbH (Mannheim, Germany, 1993) I.B.R. and in Liebl et al. (International Journal of Systematic Bacteriology (1991) 41: 255-260) I.B.R. The hybridization takes place under stringent conditions, that is to say there are formed only hybrids in which the probe and the target sequence, i.e. the polynucleotides treated with the probe, are at least 70% identical. It is known that the stringency of the hybridization, including the washing steps, is influenced or determined by varying the buffer composition, the temperature and the salt concentration. The hybridization reaction is preferably carried out with relatively low stringency as compared with the washing steps (Hybaid Hybridisation Guide, Hybaid Limited, Teddington, UK, 1996 I.B.R.).

There may be used for the hybridization reaction, for example, a 5×SSC buffer at a temperature of approximately from 50° C. to 68° C. In that case, probes may also hybridize with polynucleotides that are less than 70% identical with the sequence of the probe. Such hybrids are less stable and are removed by washing under stringent conditions. That may be achieved, for example, by lowering the salt concentration to 2×SSC and optionally subsequently to 0.5×SSC (The DIG System User's Guide for Filter Hybridisation, Boehringer Mannheim, Mannheim, Germany, 1995 I.B.R.), a temperature of approximately from 50° C. to 68° C. being set. It is optionally possible to lower the salt concentration down to 0.1×SSC. By raising the hybridization temperature stepwise from 50° C. to 68° C. in steps of approximately from 1° to 2° C., it is possible to isolate polynucleotide fragments that are, for example, at least 70% or at least 80% or at least from 90% to 95% identical with the sequence of the probe used. Further instructions for hybridization are commercially available in the form of so-called kits (e.g. DIG Easy Hyb from Roche Diagnostics GmbH, Mannheim, Germany, Catalog No. 1603558).

The person skilled in the art will find instructions on the amplification of DNA sequences with the aid of the polymerase chain reaction (PCR) inter alia in the handbook of Gait: Oligonucleotide Synthesis: A Practical Approach (IRL Press, Oxford, UK, 1984 I.B.R.) and in Newton and Graham: PCR (Spektrum Akademischer Verlag, Heidelberg, Germany, 1994).

It has been found that coryneform bacteria produce amino acids in an improved manner after overexpression of the thyA gene.

By improving the growth it is possible in particular to increase the space-time yield of the amino acid(s) formed during the fermentation.

In order to achieve overexpression, the number of copies of the corresponding genes can be increased, or the promoter and regulation region or the ribosome binding site, which is located upstream of the structural gene, can be mutated. Expression cassettes inserted upstream of the structural gene have a similar effect. By means of inducible promoters it is additionally possible to increase the expression in the course of the production of amino acids by fermentation. Expression is also improved by measures to prolong the life of the m-RNA. Furthermore, the enzyme activity is also enhanced by preventing degradation of the enzyme protein. The genes or gene constructs may either be present in plasmids with different numbers of copies or be integrated and amplified in the chromosome. Alternatively, overexpression of the genes in question may also be achieved by changing the composition of the medium and the manner in which culturing is carried out.

The person skilled in the art will find instructions thereon inter alia in Martin et al. (Bio/Technology 5, 137-146 (1987) I.B.R.), in Guerrero et al. (Gene 138, 35-41 (1994) I.B.R.), Tsuchiya and Morinaga (Bio/Technology 6, 428-430 (1988) I.B.R.), in Eikmanns et al. (Gene 102, 93-98 (1991)) I.B.R., in EP 0 472 869 I.B.R., in U.S. Pat. No. 4,601,893 I.B.R., in Schwarzer and Pühler (Bio/Technology 9, 84-87 (1991) I.B.R., in Reinscheid et al. (Applied and Environmental Microbiology 60, 126-132 (1994)) I.B.R., in LaBarre et al. (Journal of Bacteriology 175, 1001-1007 (1993)) I.B.R., in WO 96/15246 I.B.R., in Malumbres et al. (Gene 134, 15-24 (1993)) I.B.R., in JP-A-10-229891 I.B.R., in Jensen and Hammer (Biotechnology and Bioengineering 58, 191-195 (1998)) I.B.R., in Makrides (Microbiological Reviews 60:512-538 (1996)) I.B.R. and in known textbooks of genetics and molecular biology.

For the purposes of enhancement, the thyA gene of the invention was overexpressed, for example, with the aid of episomal plasmids. Suitable plasmids are those which are replicated in coryneform bacteria. Many known plasmid vectors, such as, for example, pZ1 (Menkel et al., Applied and Environmental Microbiology (1989) 64: 549-554 I.B.R.), pEKEx1 (Eikmanns et al., Gene 102:93-98 (1991) I.B.R.) or pHS2-1 (Sonnen et al., Gene 107:69-74 (1991) I.B.R.), are based on the cryptic plasmids pHM1519, pBL1 or pGA1. Other plasmid vectors, such as, for example, those which are based on pCG4 (U.S. Pat. No. 4,489,160 I.B.R.) or pNG2 (Serwold-Davis et al., FEMS Microbiology Letters 66, 119-124 (1990) I.B.R.) or pAG1 (U.S. Pat. No. 5,158,891 I.B.R.), may likewise be used.

Also suitable are those plasmid vectors with the aid of which the process of gene amplification by integration into the chromosome can be applied, as has been described, for example, by Reinscheid et al. (Applied and Environmental Microbiology 60, 126-132 (1994)) I.B.R. for the duplication or amplification of the hom-thrB operon. In that method, the complete gene is cloned into a plasmid vector that is able to replicate in a host (typically E. coli), but not in C. glutamicum. Suitable vectors are, for example, pSUP301 (Simon et al., Bio/Technology 1, 784-791 (1983) I.B.R.), pK18mob or pK19mob (Schafer et al., Gene 145, 69-73 (1994) I.B.R.), pGEM-T (Promega corporation, Madison, Wis., USA), pCR2.1-TOPO (Shuman (1994). Journal of Biological Chemistry 269:32678-32684 I.B.R.; U.S. Pat. No. 5,487,993 I.B.R.), pCR®Blunt (Invitrogen, Groningen, Netherlands; Bernard et al., Journal of Molecular Biology, 234: 534-541 (1993)) I.B.R., pEM1 (Schrumpf et al., 1991, Journal of Bacteriology 173:4510-4516 I.B.R.) or pBGS8 (Spratt et al., 1986, Gene 41: 337-342 I.B.R.). The plasmid vector containing the gene to be amplified is then transferred to the desired strain of C. glutamicum by conjugation or transformation. The method of conjugation is described, for example, in Schäfer et al. (Applied and Environmental Microbiology 60, 756-759 (1994)) I.B.R. Methods of transformation are described, for example, in Thierbach et al. (Applied Microbiology and Biotechnology 29, 356-362 (1988)) I.B.R., Dunican and Shivnan (Bio/Technology 7, 1067-1070 (1989)) I.B.R. and Tauch et al. (FEMS Microbiological Letters 123, 343-347 (1994)) I.B.R. After homologous recombination by means of a “cross-over” occurrence, the resulting strain contains at least two copies of the gene in question.

In addition, it may be advantageous for the production of L-amino acids to enhance, especially to overexpress, in addition to the thyA gene, one or more enzymes of the biosynthesis pathway in question, of glycolysis, of the anaplerotic pathway, of the citric acid cycle, of the pentose phosphate cycle, of amino acid export, and, optionally, regulatory proteins.

Accordingly, for the production of L-amino acids, in addition to enhancing the thyA gene, one or more genes selected from the group

the gene dapA coding for dihydrodipicolinate synthase (EP-B 0 197 335 I.B.R.),

the gene gap coding for glyceraldehyde-3-phosphate dehydrogenase (Eikmanns (1992), Journal of Bacteriology 174:6076-6086 I.B.R.),

the gene tpi coding for triose phosphate isomerase (Eikmanns (1992), Journal of Bacteriology 174:6076-6086

the gene pgk coding for 3-phosphoglycerate kinase (Eikmanns (1992), Journal of Bacteriology 174:6076-6086 I.B.R.),

the gene zwf coding for glucose-6-phosphate dehydrogenase (JP-A-09224661 I.B.R.),

the gene pyc coding for pyruvate carboxylase (DE-A-198 31 609 I.B.R.),

the gene mqo coding for malate quinone oxidoreductase (Molenaar et al., European Journal of Biochemistry 254, 395-403 (1998) I.B.R.),

the gene lysC coding for a feed-back resistant aspartate kinase (Accession No. P26512; EP-B-0387527 I.B.R.; EP-A-0699759 I.B.R.),

the gene lysE coding for lysine export (DE-A-195 48 222 I.B.R.),

the gene hom coding for homoserine dehydrogenase (EP-A 0131171 I.B.R.),

the gene ilvA coding for threonine dehydratase (Mockel et al., Journal of Bacteriology (1992) 8065-8072) I.B.R.) or the allele ilvA(Fbr) coding for a feed-back resistant threonine dehydratase (Möckel et al., (1994) Molecular Microbiology 13: 833-842 I.B.R.),

the gene ilvBN coding for acetohydroxy acid synthase (EP-B 0356739 I.B.R.),

the gene ilvD coding for dihydroxy acid dehydratase (Sahm and Eggeling (1999) Applied and Environmental Microbiology 65: 1973-1979 I.B.R.),

the gene zwa1 coding for the Zwa1 protein (DE: 19959328.0 I.B.R., DSM 13115),

is/are enhanced, especially overexpressed.

It may also be advantageous for the production of L-amino acids, in addition to enhancing the thyA gene, to attenuate, especially reduce the expression of, one or more genes selected from the group

the gene pck coding for phosphoenol pyruvate carboxykinase (DE 199 50 409.1 I.B.R.; DSM 13047),

the gene pgi coding for glucose-6-phosphate isomerase (U.S. Pat. No. 09/396,478 I.B.R.; DSM 12969),

the gene poxB coding for pyruvate oxidase (DE: 1995 1975.7 I.B.R.; DSM 13114),

the gene zwa2 coding for the Zwa2 protein (DE: 19959327.2 I.B.R., DSM 13113).

The term “attenuation” in this context describes the diminution or exclusion of the intracellular activity of one or more enzymes (proteins) in a microorganism that are coded for by the corresponding DNA, by, for example, using a weak promoter or using a gene or allele that codes for a corresponding enzyme having low activity, or by inactivating the corresponding gene or enzyme (protein), and optionally by combining those measures.

By the measures of attenuation, the activity or concentration of the corresponding protein is generally reduced to from 0 to 50%, from 0 to 25%, from 0 to 10% or from 0 to 5% of the activity or concentration of the wild-type protein or of the starting microorganism.

It may also be advantageous for the production of amino acids, in addition to overexpression of the thyA gene, to exclude undesired secondary reactions (Nakayama: “Breeding of Amino Acid Producing Micro-organisms”, in: Overproduction of Microbial Products, Krumphanzl, Sikyta, Vanek (eds.), Academic Press, London, UK, 1982) I.B.R.

The microorganisms produced according to the invention also form part of the invention and can be cultivated, for the purposes of the production of amino acids, continuously or discontinuously in the batch, fed batch or repeated fed batch process. A summary of known cultivation methods is described in the textbook of Chmiel (Bioprozeβtechnik 1. Einführung in die Bioverfahrenstechnik (Gustav Fischer Verlag, Stuttgart, 1991)) or in the textbook of Storhas (Bioreaktoren und periphere Einrichtungen (Vieweg Verlag, Braunschweig/Wiesbaden, 1994)) I.B.R.

The culture medium to be used must meet the requirements of the strains in question in a suitable manner. Descriptions of culture media for various microorganisms are to be found in the handbook “Manual of Methods for General Bacteriology” of the American Society for Bacteriology (Washington D.C., USA, 1981) I.B.R.

There may be used as the carbon source sugars and carbohydrates, such as, for example, glucose, saccharose, lactose, fructose, maltose, molasses, starch and cellulose, oils and fats, such as, for example, soybean oil, sunflower oil, groundnut oil and coconut oil, fatty acids, such as, for example, palmitic acid, stearic acid and linoleic acid, alcohols, such as, for example, glycerol and ethanol, and organic acids, such as, for example, acetic acid. Those substances may be used individually or in the form of a mixture.

There may be used as the nitrogen source organic nitrogen-containing compounds, such as peptones, yeast extract, meat extract, malt extract, corn steep liquor, soybean flour and urea, or inorganic compounds, such as ammonium sulfate, ammonium chloride, ammonium phosphate, ammonium carbonate and ammonium nitrate. The nitrogen sources may be used individually or in the form of a mixture.

There may be used as the phosphorus source phosphoric acid, potassium dihydrogen phosphate or dipotassium hydrogen phosphate or the corresponding sodium-containing salts. The culture medium must also contain salts of metals, such as, for example, magnesium sulfate or iron sulfate, which are necessary for growth. Finally, essential growth substances, such as amino acids and vitamins, may be used in addition to the above-mentioned substances. Suitable precursors may also be added to the culture medium. The mentioned substances may be added to the culture in the form of a single batch, or they may be fed in in a suitable manner during the cultivation.

In order to control the pH value of the culture, basic compounds, such as sodium hydroxide, potassium hydroxide, ammonia or ammonia water, or acid compounds, such as phosphoric acid or sulfuric acid, are expediently used. In order to control the development of foam, anti-foams, such as, for example, fatty acid polyglycol esters, may be used. In order to maintain the stability of plasmids, suitable substances having a selective action, such as, for example, antibiotics, may be added to the medium. In order to maintain aerobic conditions, oxygen or gas mixtures containing oxygen, such as, for example, air, are introduced into the culture. The temperature of the culture is normally from 20° C. to 45° C. and preferably from 25° C. to 40° C. The culture is continued until the maximum amount of the desired product has formed. That aim is normally achieved within a period of from 10 hours to 160 hours.

Methods of determining L-amino acids are known from the prior art. The analysis may be carried out, for example, as described in Spackman et al. (Analytical Chemistry, 30, (1958), 1190) I.B.R. by ion-exchange chromatography with subsequent ninhydrin derivation, or it may be carried out by reversed phase HPLC, as described in Lindroth et al. (Analytical Chemistry (1979) 51: 1167-1174) I.B.R.

The process of the invention is used for the production of amino acids by fermentation.

The present invention is explained in greater detail below by means of Examples.

The following microorganism was deposited as a pure culture on May 18, 2001 at the Deutsche Sammlung für Mikroorganismen und Zellkulturen (DSMZ, Braunschweig, Germany) in accordance with the Budapest treaty:

Escherichia coli DH5amcr/pEC-XK99EthyAb1ex as DSM 14309.

The isolation of plasmid DNA from Escherichia coli and all techniques for restriction, Klenow and alkaline phosphatase treatment were carried out according to Sambrook et al. (Molecular Cloning. A Laboratory Manual (1989) Cold Spring Harbour Laboratory Press, Cold Spring Harbor, N.Y., USA) I.B.R. Methods for the transformation of Escherichia coli are also described in that handbook.

The composition of common nutrient media, such as LB or TY medium, will also be found in the handbook of Sambrook et al.

### EXAMPLE 1

Preparation of a genomic cosmid gene library from Corynebacterium glutamicum ATCC 13032

Chromosomal DNA from Corynebacterium glutamicum ATCC 13032 was isolated as described in Tauch et al. (1995, Plasmid 33:168-179) I.B.R. and partially cleaved with the restriction enzyme Sau3AI (Amersham Pharmacia, Freiburg, Germany, product description Sau3AI, Code no. 27-0913-02). The DNA fragments were dephosphorylated with shrimp alkaline phosphatase (Roche Diagnostics GmbH, Mannheim, Germany, product description SAP, Code no. 1758250). The DNA of cosmid vector SuperCos1 (Wahl et al. (1987) Proceedings of the National Academy of Sciences USA 84:2160-2164) I.B.R., obtained from Stratagene (La Jolla, USA, product description SuperCos1 Cosmid Vektor Kit, Code no. 251301) I.B.R., was cleaved with the restriction enzyme XbaI (Amersham Pharmacia, Freiburg, Germany, product description XbaI, Code no. 27-0948-02 I.B.R.) and likewise dephosphorylated with shrimp alkaline phosphatase.

The cosmid DNA was then cleaved with the restriction enzyme BamHI (Amersham Pharmacia, Freiburg, Germany, product description BamHI, Code no. 27-0868-04). The cosmid DNA so treated was mixed with the treated ATCC13032 DNA, and the batch was treated with T4-DNA ligase (Amersham Pharmacia, Freiburg, Germany, product description T4-DNA ligase, Code no. 27-0870-04). The ligation mixture was then packed in phages with the aid of Gigapack II XL Packing Extract (Stratagene, La Jolla, USA, product description Gigapack II XL Packing Extract, Code no. 200217 I.B.R.).

For infection of E. coli strain NM554 (Raleigh et al. 1988, Nucleic Acid Research 16:1563-1575 I.B.R.), the cells were taken up in 10 mM MgSO₄ and mixed with an aliquot of the phage suspension. Infection and titration of the cosmid library were carried out as described in Sambrook et al. (1989, Molecular Cloning: A Laboratory Manual, Cold Spring Harbor) I.B.R., the cells being plated out on LB agar (Lennox, 1955, Virology, 1:190 I.B.R.) with 100 mg/l ampicillin. After incubation overnight at 37° C., recombinant individual clones were selected.

### EXAMPLE 2

Isolation and sequencing of the thyA gene

The cosmid DNA of an individual colony was isolated using the Qiaprep Spin Miniprep Kit (Product No. 27106, Qiagen, Hilden, Germany) according to the manufacturer's instructions, and partially cleaved with the restriction enzyme Sau3AI (Amersham Pharmacia, Freiburg, Germany, product description Sau3AI, Product No. 27-0913-02). The DNA fragments were dephosphorylated with shrimp alkaline phosphatase (Roche Diagnostics GmbH, Mannheim, Germany, product description SAP, Product No. 1758250). After separation by gel electrophoresis, cosmid fragments having a size in the range from 1500 to 2000 bp were isolated using the QiaExII Gel Extraction Kit (Product No. 20021, Qiagen, Hilden, Germany).

The DNA of sequencing vector pZero-1, obtained from Invitrogen (Groningen, Netherlands, product description Zero Background Cloning Kit, Product No. K2500-01), was cleaved with the restriction enzyme BamHI (Amersham Pharmacia, Freiburg, Germany, product description BamHI, Product No. 27-0868-04 I.B.R.). Ligation of the cosmid fragments into the sequencing vector pZero-1 was carried out as described by Sambrook et al. (1989, Molecular Cloning: A Laboratory Manual, Cold Spring Harbor) I.B.R., the DNA mixture being incubated overnight with T4 ligase (Pharmacia Biotech, Freiburg, Germany). The ligation mixture was then electroporated into E. coli strain DH5αMCR (Grant, 1990, Proceedings of the National Academy of Sciences U.S.A., 87:4645-4649 I.B.R.) (Tauch et al. 1994, FEMS Microbiol Letters, 123:343-347 I.B.R.) and plated out on LB agar (Lennox, 1955, Virology, 1:190 I.B.R.) with 50 mg/l Zeocin.

Plasmid preparation of the recombinant clones was carried out using the Biorobot 9600 (Product No. 900200, Qiagen, Hilden, Germany). Sequencing was effected by the dideoxy chain termination method of Sanger et al. (1977, Proceedings of the National Academy of Sciences U.S.A., 74:5463-5467) I.B.R. with modifications according to Zimmermann et al. (1990, Nucleic Acids Research, 18:1067) I.B.R. The “RR dRhodamin Terminator Cycle Sequencing Kit” from PE Applied Biosystems (Product No. 403044, Weiterstadt, Germany) was used. Separation by gel electrophoresis and analysis of the sequencing reaction was carried out in a “Rotiphorese NF Acrylamid/Bisacrylamid” gel (29:1) (Product No. A124.1, Roth, Karlsruhe, Germany) using the “ABI Prism 377” sequencing device from PE Applied Biosystems (Weiterstadt, Germany).

The resulting crude sequence data were then processed using the Staden program package (1986, Nucleic Acids Research, 14:217-231 I.B.R.) Version 97-0. The individual sequences of the pZero1 derivatives were assembled to a coherent contig. The computer-assisted coding region analysis was prepared using the program XNIP (Staden, 1986, Nucleic Acids Research, 14:217-231 I.B.R.). Further analyses can be carried out with the “BLAST search program” (Altschul et al., 1997, Nucleic Acids Research, 25:3389-3402 I.B.R.) against the non-redundant databank of the “National Center for Biotechnology Information” (NCBI, Bethesda, Md., USA) I.B.R.

The relative degree of substitution or mutation in the polynucleotide or amino acid sequence to produce a desired percentage of sequence identity can be established or determined by well-known methods of sequence analysis. These methods are disclosed and demonstrated in Bishop, et al. “DNA & Protein Sequence Analysis (A Practical Approach”), Oxford Univ. Press, Inc. (1997) I.B.R. and by Steinberg, Michael “Protein Structure Prediction” (A Practical Approach), Oxford Univ. Press, Inc. (1997) I.B.R.

The resulting nucleotide sequence is shown in SEQ ID No. 1. Analysis of the nucleotide sequence gave an open reading frame of 801 base pairs, which was designated the thyA gene. The thyA gene codes for a protein of 266 amino acids.

### EXAMPLE 3

Preparation of the shuttle expression vector pEC-XK99EthyAb1ex for enhancement of the thyA gene in C. glutamicum

3.1 Cloning of the thyA gene

Chromosomal DNA was isolated from the strain ATCC 13032 by the method of Eikmanns et al. (Microbiology 140: 1817-1828 (1994)) I.B.R. On the basis of the sequence of the thyA gene known from Example 2 for C. glutamicum, the following oligonucleotides were selected for the polymerase chain reaction (see SEQ ID No. 3 and SEQ ID No. 4):

thyAex1:

5′ ca ggt acc-tga cgg cat gac tgt tcc aa 3′ SEQ ID NO: 3

thyAex2:

5′ gt tct aga-acc gat cat acg gcg acc tt 3′ SEQ ID NO: 4

The primers shown were synthesised by MWG-Biotech AG (Ebersberg, Germany) and the PCR reaction was carried out according to the standard PCR method of Innis et al. (PCR protocols. A guide to methods and applications, 1990, Academic Press) I.B.R. with Pwo polymerase from Roche Diagnostics GmbH (Mannheim, Germany). With the aid of the polymerase chain reaction, the primers permit amplification of a DNA fragment 829 bp in size that carries the thyA gene. In addition, the primer thyAex1 contains the sequence for the cleavage site of the restriction endonuclease KpnI, and the primer thyAex2 contains the cleavage site of the restriction endonuclease XbaI, which are indicated in the above nucleotide sequence by underlining.

The thyA fragment 829 bp in size was cleaved with the restriction endonucleases KpnI and XbaI and then isolated from the agarose gel using the QiaExII Gel Extraction Kit (Product No. 20021, Qiagen, Hilden, Germany).

3.2 Construction of the shuttle vector pEC-XK99E

The E. coli-C. glutamicum shuttle vector pEC-XK99E was constructed according to the prior art. The vector contains the replication region rep of plasmid pGA1 including the replication effector per (U.S. Pat. No. 5,175,108 I.B.R.; Nesvera et al., Journal of Bacteriology 179, 1525-1532 (1997) I.B.R.), the kanamycin resistance gene aph(3′)-IIa from Escherichia coli (Beck et al. (1982), Gene 19: 327-336 I.B.R.), the origin of replication, the trc promoter, the termination regions T1 and T2, the lacI^{q} gene (repressor of the lac operon of E. coli) and a multiple cloning site (mcs) (Norrander, J. M. et al. Gene 26, 101-106 (1983) I.B.R.) of plasmid pTRC99A (Amann et al. (1988), Gene 69: 301-315 I.B.R.).

The constructed E. coli-C. glutamicum shuttle vector pEC-XK99E was transferred to C. glutamicum DSM5715 by means of electroporation (Liebl et al., 1989, FEMS Microbiology Letters, 53:299-303 I.B.R.). Selection of the transformants was carried out on LBHIS agar consisting of 18.5 g/l brain-heart infusion broth, 0.5 M sorbitol, 5 g/l Bacto tryptone, 2.5 g/l Bacto yeast extract, 5 g/l NaCl and 18 g/l Bacto agar, which had been supplemented with 25 mg/l kanamycin. Incubation was carried out for 2 days at 33° C.

Plasmid DNA was isolated from a transformant by the conventional methods (Peters-Wendisch et al., 1998, Microbiology, 144, 915-927 I.B.R.), cleaved with the restriction endonuclease HindIII, and the plasmid was examined by subsequent agarose gel electrophoresis.

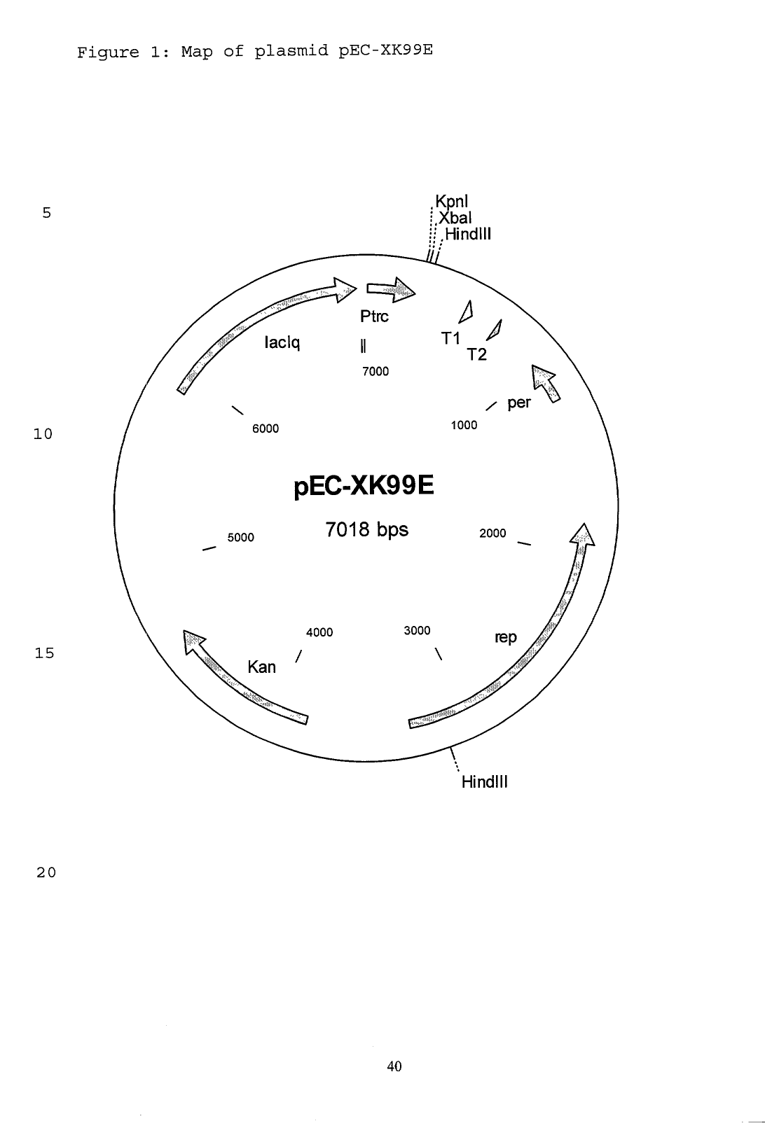

The plasmid construct so obtained was designated pEC-XK99E (FIG. 1). The strain obtained by electroporation of plasmid pEC-XK99E into C. glutamicum strain DSM5715 was named DSM5715/pEC-XK99E and deposited as DSM13455 at the Deutsche Sammlung fur Mikroorganismen und Zellkulturen (DSMZ, Braunschweig, Germany) in accordance with the Budapest treaty.

3.3 Cloning of thyA into the E. coli-C. glutamicum shuttle vector pEC-XK99E

The E. coli-C. glutamicum shuttle vector pEC-XK99E described in Example 3.2 was used as the vector. DNA of that plasmid was cleaved completely with the restriction enzymes KpnI and XbaI and then dephosphorylated with shrimp alkaline phosphatase (Roche Diagnostics GmbH, Mannheim, Germany, product description SAP, Product No. 1758250).

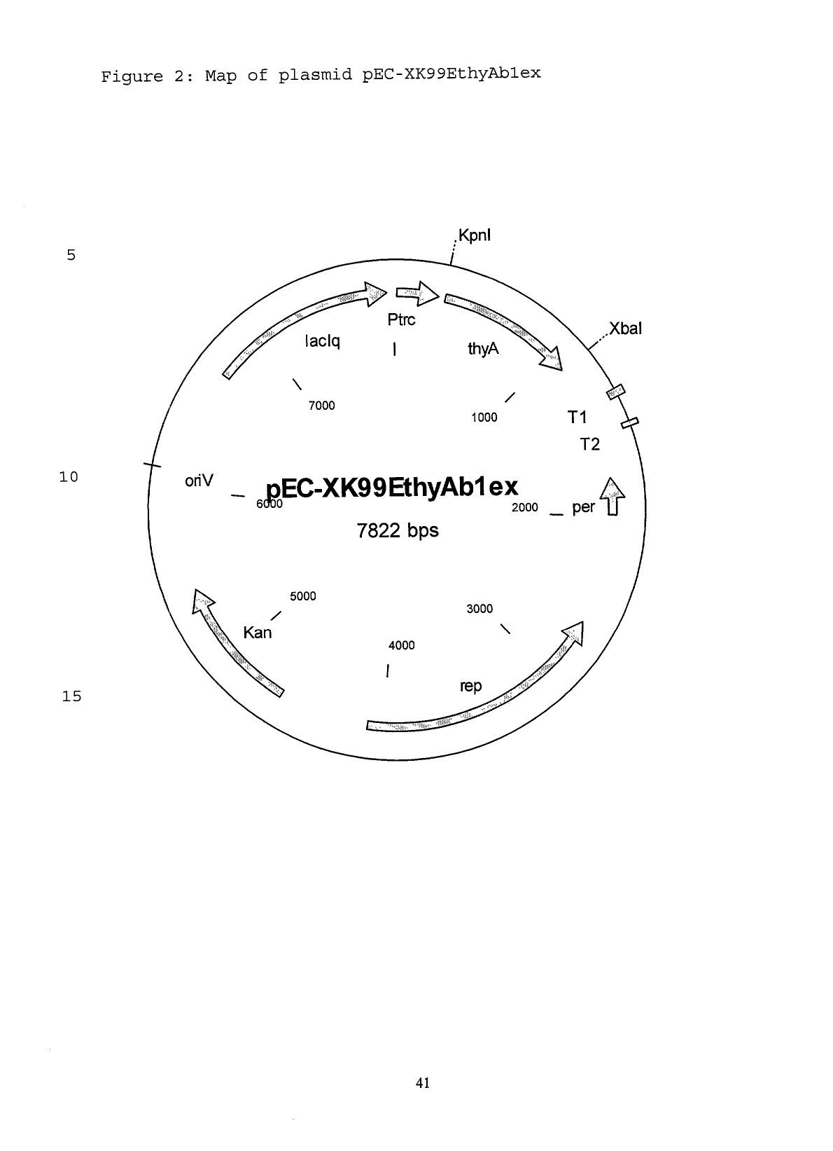

The thyA fragment approximately 815 bp in size, described in Example 3.1, obtained by means of PCR and cleaved with the restriction endonucleases KpnI and XbaI, was mixed with the prepared vector pEC-XK99E and the batch was treated with T4-DNA ligase (Amersham Pharmacia, Freiburg, Germany, product description T4-DNA ligase, Code no. 27-0870-04 I.B.R.). The ligation batch was transformed into E. coli strain DH5αmcr (Hanahan, in: DNA Cloning. A Practical Approach. Vol. I, IRL-Press, Oxford, Washington D.C., USA I.B.R.). The selection of plasmid-carrying cells was effected by plating out the transformation batch on LB agar (Lennox, 1955, Virology, 1:190 I.B.R.) with 50 mg/l kanamycin. After incubation overnight at 37° C., recombinant individual clones were selected. Plasmid DNA was isolated from a transformant using the Qiaprep Spin Miniprep Kit (Product No. 27106, Qiagen, Hilden, Germany) according to the manufacturer's instructions and was cleaved with the restriction enzymes XbaI and KpnI in order to examine the plasmid by subsequent agarose gel electrophoresis. The resulting plasmid was named pEC-XK99EthyAb1ex. It is shown in FIG. 2.

### EXAMPLE 4

Transformation of strain DSM5715 with the plasmid pEC-XK99EthyAb1ex

Strain DSM5715 was transformed with the plasmid pEC-XK99EthyAb1ex using the electroporation method described by Liebl et al., (FEMS Microbiology Letters, 53:299-303 (1989)) I.B.R. Selection of the transformants was carried out on LBHIS agar consisting of 18.5 g/l brain-heart infusion broth, 0.5 M sorbitol, 5 g/l Bacto tryptone, 2.5 g/l Bacto yeast extract, 5 g/l NaCl and 18 g/l Bacto agar, which had been supplemented with 25 mg/l kanamycin. Incubation was carried out for 2 days at 33° C.

Plasmid DNA was isolated from a transformant by the conventional methods (Peters-Wendisch et al., 1998, Microbiology, 144, 915-927 I.B.R.), cleaved with the restriction endonucleases XbaI and KpnI, and the plasmid was examined by subsequent agarose gel electrophoresis. The resulting strain was named DSM5715/pEC-XK99EthyAb1ex1.

### EXAMPLE 5

Production of Lysine

The C. glutamicum strain DSM5715/pEC-XK99EthyAb1ex obtained in Example 4 was cultivated in a nutrient medium suitable for the production of lysine, and the lysine content in the culture supernatant was determined.

To that end, the strain was first incubated for 24 hours at 33° C. on agar plate with the corresponding antibiotic (brain-heart agar with kanamycin (25 mg/l)). Starting from that agar plate culture, a pre-culture was inoculated (10 ml of medium in 100 ml Erlenmeyer flasks). CgIII complete medium was used as the medium for the pre-culture.

Cg III medium

| | | |
|---|---|---|
| NaCl | 2.5 | g/l |
| Bacto peptone | 10 | g/l |
| Bacto yeast extract | 10 | g/l |
| Glucose (autoclaved separately) | 2% | (w/v) |

The pH value was adjusted to pH 7.4

Kanamycin (25 mg/l) was added thereto. The pre-culture was incubated for 16 hours at 33° C. at 240 rpm on a shaker. A main culture was inoculated from that pre-culture, so that the initial OD (660 nm) of the main culture was 0.1. MM medium was used for the main culture.

MM medium

| | | |
|---|---|---|
| CSL (corn steep liquor) | 5 | g/l |
| MOPS (morpholinopropane sulfonic | 20 | g/l |
| acid) | | |
| Glucose (autoclaved separately) | 50 | g/l |
| (NH₄)₂SO₄ | 25 | g/l |
| KH₂PO₄ | 0.1 | g/l |
| MgSO₄ * 7H₂O | 1 | g/l |
| CaCl₂ * 2H₂O | 10 | mg/l |
| FeSO₄ * 7H₂O | 10 | mg/l |
| MnSO₄ * H₂O | 5 | mg/l |
| Biotin (sterilized by filtration) | 0.3 | mg/l |
| Thiamin * HCl (sterilized by | 0.2 | mg/l |
| filtration) | | |
| L-Leucine (sterilized by | 0.1 | g/l |
| filtration) | | |
| CaCO₃ | 25 | g/l |

CSL, MOPS and the salt solution were adjusted to pH 7 with ammonia water and autoclaved. The sterile substrate and vitamin solutions were then added, as well as the dry autoclaved CaCO₃.

Cultivation was carried out in a volume of 10 ml in a 100 ml Erlenmeyer flask with baffles. Kanamycin (25 mg/l) was added. Cultivation was carried out at 33° C. and 80% humidity.

After 48 hours, the OD was determined at a measuring wavelength of 660 nm using a Biomek 1000 (Beckmann Instruments GmbH, Munich). The amount of lysine that had formed was determined using an amino acid analyzer from Eppendorf-BioTronik (Hamburg, Germany) by ion-exchange chromatography and post-column derivation with ninhydrin detection.

The result of the test is shown in Table 1.

TABLE 1

| | OD | Lysine HCl |
|---|---|---|
| Strain | (660 nm) | g/l |
| DSM5715 | 11.3 | 13.02 |
| DSM5715/pEC- | 13.3 | 14.00 |
| XK99EthyAblex | | |

This application claims priority to German Priority Document Application No. 100 46 626.5, filed on Sep. 20, 2000 and to German Priority Document Application No. 101 33 162.2, filed on Jul. 7, 2001. Both German Priority Documents are hereby incorporated by reference in their entirety.

We claim:

1. An isolated polynucleotide from coryneform bacteria, containing a polynucleotide sequence coding for the thyA gene, selected from the group consisting of:
a) a polynucleotide that is at least 70% identical with a polynucleotide that codes for a polypeptide containing the amino acid sequence of SEQ ID No. 2,
b) a polynucleotide that codes for a polypeptide containing an amino acid sequence that is at least 70% identical with the amino acid sequence of SEQ ID No. 2,
c) a polynucleotide that is complementary to the polynucleotides of a) or b), and
d) a polynucleotide containing at least 15 consecutive nucleotides of the polynucleotide sequence of a), b) or c),

2. The polynucleotide according to claim 1, wherein the polypeptide has thymidilate synthase activity.

3. The polynucleotide as claimed in claim 1, wherein the polynucleotide is a recombinant DNA, that is replicable in coryneform bacteria.

4. The polynucleotide as claimed in claim 1, wherein the polynucleotide is an RNA.

5. The polynucleotide as claimed in claim 3, comprising the nucleic acid sequence as shown in SEQ ID No. 1.

6. The polynucleotide according to claim 3, wherein the DNA, comprises
(i) the nucleotide sequence shown in SEQ ID No. 1, or
(ii) at least one sequence that corresponds to sequence (i) within the region of the degeneracy of the genetic code, or
(iii) at least one sequence that hybridizes with the sequence that is complementary to sequence (i) or (ii).

7. The polynucleotide according to claim 6, further comprising
(iv) sense mutations in (i) that are neutral in terms of function.

8. The polynucleotide according to claim 6, wherein the hybridization of sequence (iii) is carried out under conditions of stringency corresponding at most to 2×SSC.

9. The polynucleotide sequence according to claim 1, wherein the polynucleotide codes for a polypeptide that comprises the amino acid sequence shown in SEQ ID NO: 2.

10. A coryneform bacterium in which the thyA gene is enhanced.

11. The coryneform bacterium according to claim 10, wherein the thyA gene is overexpressed.

12. An Escherichia coli DH5amcr/pEC-XK99EthyAb1ex deposited as DSM 14309.

13. A method for the production of L-amino acids, by fermentation in coryneform bacteria, comprising:
a) fermenting, in a medium, the coryneform bacteria producing the desired L-amino acid, in which bacteria at least the thyA gene or nucleotide sequences coding therefor are enhanced.

14. The method according to claim 13, further comprising:
b) concentrating the L-amino acid in the medium or in the cells of the bacteria.

15. The method according to claim 14, further comprising:
c) isolating the L-amino acid.

16. The method according to claim 13, wherein the L amino acids are lysine.

17. The method according to claim 13, wherein the thyA gene or nucleotide sequences coding for this gene are overexpressed.

18. The method according to claim 13, wherein additional genes of the biosynthesis pathway of the desired L-amino acid are enhanced in the bacteria.

19. The method according to 13, wherein bacteria are used in which at least some of the metabolic pathways that reduce formation of the desired L-amino acid are excluded.

20. The method according to claim 13, wherein the bacteria are transformed with a plasmid vector and the plasmid vector carries the nucleotide sequence coding for the thyA gene.

21. The method according to claim 13, wherein expression of the polynucleotide(s) coding for the thyA gene is enhanced.

22. The method according to claim 21, wherein expression of the polynucleotide(s) coding for the thyA gene is overexpressed.

23. The method according to claim 13, wherein the catalytic properties of the polypeptide for which the polynucleotide thyA codes are increased.

24. The method according to claim 13, wherein the bacteria being fermented comprise, at the same time, one or more genes which are enhanced or overexpressed; wherein the one or more genes is/are selected from the group consisting of:
the gene dapA coding for dihydrodipicolinate synthase,
the gene gap coding for glyceraldehyde-3-phosphate dehydrogenase,
the gene tpi coding for triose phosphate isomerase,
the gene pgk coding for 3-phosphoglycerate kinase,
the gene zwf coding for glucose-6-phosphate dehydrogenase,
the gene pyc coding for pyruvate carboxylase,
the gene mqo coding for malate quinone oxidoreductase,
the gene lysC coding for a feed-back resistant aspartate kinase,
the gene lysE coding for lysine export,
the gene hom coding for homoserine dehydrogenase,
the gene ilvA coding for threonine dehydratase or the allele ilvA(Fbr) coding for a feed-back resistant threonine dehydratase,
the gene ilvBN coding for acetohydroxy acid synthase,
the gene ilvD coding for dihydroxy acid dehydratase, and
the gene zwa1 coding for the Zwa1 protein.

25. The method according to claim 13, wherein the bacteria being fermented comprise, at the same time, one or more genes which are attenuated; wherein the genes are selected from the group consisting of:
the gene pck coding for phosphoenol pyruvate carboxykinase,
the gene pgi coding for glucose-6-phosphate isomerase,
the gene poxB coding for pyruvate oxidase, and
the gene zwa2 coding for the Zwa2 protein.

26. The method according to claim 13, wherein microorganisms of the species Corynebacterium glutamicum are used.

27. The method according to claim 26, wherein the Corynebacterium strain DSM5715/pEC-XK99EthyAb1ex is used.

28. A coryneform bacterium comprising a vector that carries a polynucleotide as claimed in claim 1.

29. A method of finding RNA, cDNA and DNA in order to isolate nucleic acids, or polynucleotides or genes, that code for thymidilate synthase or are very similar to the sequence of the thyA gene, comprising contacting the RNA, cDNA, or DNA with hybridization probes comprising polynucleotide sequences according to claim 1.

30. The method according claim 29, wherein arrays, micro arrays or DNA chips are used.


SEQUENCE LISTING (4 sequences)

SEQ ID NO: 1 - DNA, 1200 bp, Corynebacterium glutamicum
  CDS (231)..(1028)
tccacgccggcggccaatacgagtgggattccgcagcaccagtcggcgtctgcaaggcagcaggcttgcactgctccaggctcgacggttccgagctgacctacaacaacaaagacacctacatgccagacatcttgatctgtcgccctgaacttgcagatgaacttctcgagatgtgcgcgaagttctacgaggagaatggaacttactaacgctgttatgatgacggcatgactgttccaacgccttatgaagaccttcttcggaagattgctgaagaagggtcccacaaggacgaccgcaccggcaccggcactacttctttattcggacaacaaatccgctttgatctcaatgaaggttttccccttctgaccaccaagaaggtccatttccactctgttgtgggtgagcttttgtggttccttcagggggattccaacgtcaaatggctgcaggataacaacatccgcatttggaatgaatgggcagatgaggacggcgagctgggccctgtttatggtgtccagtggcgttcttggccaacccctgatggtcgtcacattgaccagatctcaggtgctttagaaactctgcgaaacaaccctgattcacgtcgcaatattgtctcggcgtggaatgtttccgagcttgaaaacatggctcttcccccttgtcacttgcttttccagctctatgtcgccgatggcaaactgtcttgccagctctaccagcgttctgcggacatgttcctgggtgtgcctttcaacatcgcatcttatgcactgctcacccacatgtttgcccagcaggcaggcttggaagtcggcgagttcatttggactggcggcgactgccacatttatgacaaccacaaggaacaggtcgcggagcagctgagccgcgaagctcgcccctaccccaccttggagctcaacaaggcagcgtccatgtttgagtacagcttcgatgacatcaccgtgtccggctacgatccacacccattgatccgcggcaaggtcgccgtatgatcggtgcgatttgggcacaaggccgcgacggcatcatcggcgacggcaccgacatgccctggcacatcccggaagacctcaaacacttcaagaaaaccaccatgggccagccggtcatcatgggtcgtcgcacgtgggagtctttgccgttcaagccgcttcccggccg

SEQ ID NO: 2 - protein, 266 aa, Corynebacterium glutamicum
MTVPTPYEDLLRKIAEEGSHKDDRTGTGTTSLFGQQIRFDLNEGFPLLTTKKVHFHSVVGELLWFLQGDSNVKWLQDNNIRIWNEWADEDGELGPVYGVQWRSWPTPDGRHIDQISGALETLRNNPDSRRNIVSAWNVSELENMALPPCHLLFQLYVADGKLSCQLYQRSADMFLGVPFNIASYALLTHMFAQQAGLEVGEFIWTGGDCHIYDNHKEQVAEQLSREARPYPTLELNKAASMFEYSFDDITVSGYDPHPLIRGKVAV

SEQ ID NO: 3 - DNA, 28 bp, Corynebacterium glutamicum
caggtacctgacggcatgactgttccaa

SEQ ID NO: 4 - DNA, 28 bp, Corynebacterium glutamicum
gttctagaaccgatcatacggcgacctt